(19) United States
(12) Patent Application Publication
(10) Pub. No.: US 2009/0326329 A1
(43) Pub. Date: Dec. 31, 2009

(54) BOX FOR ENDOSCOPE

(75) Inventor: Kenji YAMANE, Saitama-shi (JP)

Correspondence Address: BIRCH STEWART KOLASCH & BIRCH, PO BOX 747, FALLS CHURCH, VA, 22040-0747, US

(73) Assignee: FUJIFILM CORPORATION, Tokyo (JP)

(21) Appl. No.: 12/490,551

(22) Filed: Jun. 24, 2009

(30) Foreign Application Priority Data
Jun. 27, 2008 (JP) 2008-169585
Jun. 27, 2008 (JP) 2008-169586
Jun. 27, 2008 (JP) 2008-169587
Jun. 27, 2008 (JP) 2008-169588

(51) Int. Cl. A61B 1/06

(52) U.S. Cl. 600/178

(57) ABSTRACT

In a light source box disposed to project in a predetermined direction from an endoscope operation section and mounting a rear supporting body on a front supporting body, a cylindrical conductive body forming a notch is disposed in a battery storage section of the rear supporting body. On the rotating switch disposed on the rear end of the rear supporting body, an engagement section is mounted which integrates a resilient connector for the battery connection and a switch movable connector disposed up to the inner side face of the battery storage section. The movable connector is placed in an OFF position by displacement to the notch and is placed in an ON position by connect with the side face of the cylindrical conductive body due to rotational operation of the rotating switch.

The disclosure of Japanese Patent Applications Nos. 2008-169585, 2008-169586, 2008-169587 and 2008-169588, filed on Jun. 27, 2008, including its specification, claims and drawings, is incorporated herein by reference in its entirety.

### BACKGROUND OF THE INVENTION

1. Field of the Invention

The present invention relates to a battery box or light source box for an endoscope. In particular, the present invention relates to a structure in a functional box projecting from an operation section such as a portable endoscope, storing a battery acting as a power source and functioning for example as a light source.

2. Description of the Related Art

An endoscope is a device using a light source to illuminate an observed object with light from a tip section of the endoscope (scope) in order to observe the illuminated object optically or electronically by use of the solid-state image sensor etc. Development of portable endoscopes has been promoted in recent years to enable use in settings other than those provided with equipment, use at the bed side and during emergencies. Such portable endoscopes dispose a battery and an LED light source in a box provided in an operation section of the endoscope.

A conventional portable endoscope is shown in FIG. 18. The endoscope includes a tip section 1A, a curved section and flexible section 1B and an operation section 1C. A forceps port 2 and an eye piece (objective optical system) 3 are provided in the operation section 1C. A light source box (also functioning as battery box) 4 storing a battery is provided in a vertical orientation with respect to the axial dimension of the endoscope.

FIG. 19 shows the structure of the battery section towards the rear end of the light source box 4. A rotating switch 5 is disposed to rotate through a fixed angle on the rear end of the main body 4B of the optical source box 4. A battery 7 is disposed in a battery storage section in an internal section of the main body 4B. On the rear end of the main body 4B, a conductive section 9 is mounted to contact the negative (−) electrode of the battery 7 via a coil spring 8 and a fixed connector 9a is provided on the conductive section 9. A switch movable connector 10 is mounted on the rotating switch 5 and is connected to the light source drive circuit through the main body 4b.

FIG. 20 shows the structure of the light source towards the front end of the light source box 4. The light source box 4 is provided with a front supporting body 4a and a rear supporting body 4b. A light source drive circuit base 308 is provided on the front supporting body 4a. The base 308 mounts an LED section 307 disposing an LED which is a light source and a circuit for driving the LED of the LED section 307, for example an LED drive circuit 308a including a constant current power supply and a booster circuit. In other words, the LED section 307 is fixed to the front supporting body 4a by a fixing screw 310. The light source drive circuit base 308 is fixed by the screw 310 through a spacer 309 to the LED section 307. A battery 7 is disposed on and connected to the rear face of the light source drive circuit base 308 via a battery connection connector 308b for the positive electrode.

In this type of light source box 4, when the rotating switch 5 is rotated through a fixed angle, the switch movable connector 10 makes contact with the fixed connector 9a thereby supplying power from the battery 7 to the light source and illuminating the LED section 7 by driving the LED drive circuit 308a.

### SUMMARY OF THE INVENTION

However as shown in FIG. 18, the light source box 4 storing the battery 7 of a conventional portable endoscope projects vertically from operation section 1C with respect to the axial dimension of the endoscope. Consequently when there is a large amount of overhang (projection amount) L and the outer diameter is large, the problem arises that the light source box 4 interferes with operation of the endoscope by an operator. In other words, an operator must operate the device while holding the section between the forceps port 2 and the light source box 4 of the operation section 1C with one hand. When the light source box 4 is long and the outer diameter is large, it comes into contact with the chest of the operator and the rotational moment of the center axis of the endoscope increases thereby causing an adverse effect on operability. Thus there is a need for a light source box 4 which has an extremely short overhang L and a small outer diameter.

Furthermore the prior art includes examples in which the amount of overhang L is reduced by use of an extremely short battery. However the capacity of the battery is reduced corresponding to the reduction in length which causes the inconvenience of increasing the frequency of exchanging the battery during use (observation or processing). Furthermore since the time for observation or processing using a portable endoscope differs depending on the purpose, it is sometimes the case that a short battery with a small capacity may be sufficient and thus it is convenient to select a battery capacity in response to the use time.

Furthermore as disclosed in Patent Document 2 (Japanese Patent Application Publication No. JP-A-2000-56239), the prior art includes devices in which an auxiliary power source unit to enable long time observation provided with an auxiliary battery is mounted to be freely detachable with respect to the battery-operated light source storing the battery. However the structure in this case is complicated by the addition of the auxiliary power source unit as a separate component.

As described in FIG. 20, in a conventional light source box 4, the LED section (light source section) 307 and the light source drive circuit base 308 which are separate components are fixed respectively using the screw 310 and the light source drive circuit base 308 is mounted using the spacer 309. The mounting of these two members is complicated and the problem of handling difficulties arises during maintenance and exchanging operations. Moreover when the outer periphery of each member 307, 308 is fixed by a plurality of screws 310, the outer diameter of the light source box (supporting body 4a) itself is increased by the region required for connection and mounting.

Furthermore since a conventional endoscope including the light source box 4 is washed and disinfected for the purpose of prevention of infection after use, the light source box 4 is provided with a water-tight structure using an O ring for example in the connection section of the front supporting body 4a and the rear supporting body 4b to thereby maintain the internal section in a water-tight state.

However a user must perform battery exchange of the light source box 4 in the conventional example and when the mounting connection of the rear supporting body (separating section) 4b with respect to the front supporting body (base) 4a is incomplete, the problem arises that water-tight conditions in the internal section cannot be maintained. Although prior-art examples attempt to maintain water-tight conditions by the provision of a mark on the front supporting body 4a indicating the fixing position of the rear supporting body 4b during connection operations, this type of method may result in incomplete connection of the front supporting body 4a and the rear supporting body 4b. In this case, moisture enters into the light source box 4, washing and disinfection is insufficient in addition to the fact that the inner electrical members undergo corrosion.

The present invention is proposed to solve the above problems and has a first object of providing a box for endoscope in which the amount of overhang from the endoscope operation section is reduced and which maintains good operation characteristics of the endoscope.

A second object is to enable selection of a battery having a capacity corresponding to the use time of the endoscope and moreover to simplify the selection of the battery and reduce the amount of overhang from the endoscope operation section.

A third object is to facilitate the mounting and therefore the handling during maintenance or exchange of the light source drive circuit base and the light source section and furthermore to maintain good operation characteristics of the endoscope by reducing the outer diameter of the box itself.

A fourth object is to ensure execution of the washing and disinfection by an arrangement which always ensures the water-tight state of the internal section when use is enabled by connecting a separating section to a base.

In order to achieve the first object above, the present invention is a box for endoscope (battery box) disposed to project in a predetermined direction from the endoscope operation section and which is provided with a rotating switch (power input switch, drive commencement switch) and battery storage section storing a battery. The box for endoscope is provided with a resilient connector for the battery connection disposed on a rear end of the box main body, a switch movable connector connected to the resilient connector, disposed up to the inner face of the battery storage section (side face of the battery) and fixed to the rotating switch, and a switch fixed conductive body disposed on the inner face (inner peripheral face) of the battery storage section. The switch operation is executed by contact/breaking contact of the switch movable connector and the switch fixed conductive body in response to the rotation operation of the rotating switch.

The switch fixed conductive body is provided with a notch on a peripheral section of the conductive cylindrical body. The switch movable connector is placed in an OFF position when displaced to the notch and is placed in an ON position when contacting with the side face of the cylindrical body.

According to this type of structure, when the rotating switch is rotated in an ON direction, the movable connector for example makes contact with the side face of the fixed conductive body formed from a cylindrical body and is placed in the ON position. When the rotating switch is rotated in an OFF direction, the movable connector is displaced to the notch of the cylindrical fixed conductive body and the movable connector is placed in the OFF position.

In the box for endoscope according to the present invention, the switch movable connector is disposed towards the side face of the battery (the cylindrical outer side face) and since the switching operation is performed in the interval with the fixed conductive body disposed in this section, there is no need for a space for both switch connectors present on the rear end of a conventional light source box. Furthermore a coil spring is not used. Thus the amount of overhang from the endoscope operation section of the battery box (or light source box) is shortened and good operation of the endoscope is maintained.

In order to achieve the second object, in a further invention, a box for endoscope is disposed to project in a predetermined direction from the endoscope operation section and stores a battery. The box forms a box main body including a front supporting body mounted on the operation section and a rear supporting body freely detachable from the front supporting body. The rear supporting body has battery storage section of a size enabling the storage of respective types of batteries of differing sizes and a plurality of rear supporting bodies are provided in common with the connection sections for the front supporting bodies.

According to this further invention, since a plurality of rear supporting bodies are provided with a size adapted to various batteries of differing sizes (capacities) and common connection sections are provided as connection sections of the rear supporting bodies with respect to the front supporting bodies, a battery having a capacity corresponding to the length of time for use (observation or processing) of the endoscope can be mounted and used. The further invention enables the selection of a battery having a capacity corresponding to the use time of the endoscope. Moreover the effect is obtained that selection is enabled with a simplified structure and one in which overhang from the operation section of the endoscope is shortened.

The structure of the further invention enables storage of batteries of different diameters by adjusting the wall thickness of the cylindrical conductive body when providing the movable connector and the cylindrical conductive body forming a notch. In this manner, the advantage is obtained that storage of batteries of varying diameter is ensured with respect to the size of the diameter of a battery by simply varying the wall thickness of the cylindrical body or the cylindrical conductive body without the need to vary the thickness of the side face of the rear supporting body.

In order to achieve the third object, in yet a further invention, a box for endoscope is disposed to project in a predetermined direction from the endoscope operation section and mounts a light source section retaining a light source for illumination and a light source drive circuit driving the light source. The light source section and the light source drive circuit are mounted on a single assembly holder and the assembly holder is fixed to the box main body.

According to this yet further invention, the light source section and the light source drive circuit are mounted by a screw or adhesive to the cylindrical assembly holder and fixed to the main body as a single assembly. The affixation can be performed with a pressing ring provided with a male threaded section on an outer periphery. The male threaded section of the pressing ring is threadably engaged with a female threaded section of the inner section of the supporting body and the pressing ring presses a ring-shaped step of the holder in order to fix the assembly. Thus the light source section and the light source drive circuit can be handled as a single assembly and thus facilitates handling during maintenance and exchanging operation as well as mounting of the light source section and the light source drive circuit.

In this yet further invention, the assembly holder is formed as a cylindrical body forming a ring-shaped step which has a smaller outer diameter on the rear side. The pressing ring forms a threaded section. Thus by engaging the pressing ring to the threaded section on the supporting body side while abutting with the ring-shaped step, it is possible to fix the assembly holder to the box main body. In this manner, since the ring-shaped step of the cylindrical holder is pressed with the pressing ring without the adoption of a method of fixing the outer periphery of each member using a screw, the outer diameter of the light source box itself is reduced and it is possible to ensure good operability of the endoscope.

In order to achieve the fourth object, in yet a further invention, a box for endoscope in which a box-shaped main body is disposed in the operation section of the endoscope and is provided with a base and a separating section. The box is provided with a water-tight structure provided on an engaging section for the separating section and the base to maintain the interior of the box-shaped main body in a water-tight state and a connection structure for the conductive body connection point connecting two conductive body connection points to maintain an electrical path connected to the functional switch when the separating section is mounted on the base. A connection means is provided on the engaging section to move the separating section forward and mount and fix the separating section to the base. A distance D₁ is defined as the distance to the water-tight commencement position of the water-tight structure which is forward of a final forward motion position at which the separating section completes forward motion due to the connection means with respect to the base and a distance D₂ is defined as the distance to the connection point contact commencement position of the conductive body connection structure which is forward of the final forward motion position. Herein the setting D₂<D₁ is performed.

In this arrangement, a water-tight structure using an O ring is provided on the engaging section for the base and the separating section, and a connection means is provided which is engaged by the threadable engagement of a male threaded section and a female threaded section. The separating section is moved forward and mounted and fixed to the base by threadable engagement of the male threaded section provided on the separating section with the female threaded section provided on the base. When the separating section is moved forward during the mounting and fixing operation, firstly it reaches the water-tight commencement position of the water-tight structure and then reaches the connection point contact commencement position of the conductive body connection structure. The two connection points of the conductive body do not make connection in the absence of a water-tight state.

According to this yet further invention, when the separating section is connected with respect to the base and the functional switch is placed in an operational condition, since water-tight conditions are always maintained in the internal section, incomplete connection due to entry of water is avoided and it is possible to ensure washing and disinfection of the endoscope including the box and to prevent corrosion of internal electrical members.

### BRIEF DESCRIPTION OF THE DRAWINGS

FIG. 1 is a sectional view showing the structure of an endoscopic light source box according to a first embodiment of the present invention;

FIG. 2 is an exploded perspective view showing the main components of the light source box according to the first embodiment;

FIG. 3 is an exploded perspective view showing the members forming the light source box according to the first embodiment;

FIG. 4 is a perspective view showing the members forming the light source box when engaged according to the first embodiment as shown in FIG. 2;

FIG. 5A is a perspective view showing separation (separation of the supporting body) of the light source box when engaged according to the first embodiment;

FIG. 5B is a perspective view showing engaged state of FIG. 5A;

FIG. 6A shows the operation of the switch movable connector with respect to the cylindrical conductive body in the first embodiment and shows the rotating switch in the OFF position;

FIG. 6B shows the rotating switch in the ON position;

FIG. 7A is a sectional view showing the structure for a small battery for a light source box for an endoscope according to a second embodiment of the present invention;

FIG. 7B is a sectional view showing the structure for a large battery for a light source box for an endoscope according to the second embodiment of the present invention;

FIG. 8A is an exploded perspective view showing the main members in a light source box for a small battery according to the second embodiment of the present invention;

FIG. 8B is an exploded perspective view showing the main members in a light source box for a large battery;

FIG. 9A is a perspective view showing separation (separation of the supporting body) of the light source box for a small battery according to the second embodiment;

FIG. 9B is a perspective view showing the engaged state of a light source box for a small battery according to the second embodiment;

FIG. 10A is a perspective view showing separation of the light source box for a large battery according to the second embodiment;

FIG. 10B is a perspective view showing the engaged state of a light source box for a large battery according to the second embodiment;

FIG. 11 is a sectional view showing the structure of an endoscopic light source box according to a third embodiment of the present invention;

FIG. 12 is an exploded perspective view showing the members forming the light source assembly disposed in the light source box according to the third embodiment;

FIG. 13 is an exploded perspective view showing the structure for fixing the light source assembly to the front supporting body according to the third embodiment;

FIG. 14 is an exploded perspective view showing the structure of the engaging section of a light source box for an endoscope according to a fourth embodiment;

FIG. 15 is a sectional view showing the overall structure of the light source box according to the fourth embodiment;

FIG. 16A is a sectional view showing the relationship of the water-tight state of the water-tight structure and the connection point contact state of the conductive body connection point contact structure in a state prior to contact according to the fourth embodiment;

FIG. 16B is a sectional view showing contact of the connection points of the conductive body connection point contact structure according to the fourth embodiment;

FIG. 17 is an exploded perspective view showing the main components of the light source box according to the fourth embodiment;

FIG. 18 is a perspective view of the overall structure of a conventional endoscope;

FIG. 19 is a sectional view showing the structure of the battery section of a conventional light source box; and

FIG. 20 is a sectional view showing the structure of the light source section of a conventional light source box.

### DETAILED DESCRIPTION OF THE PREFERRED EMBODIMENTS

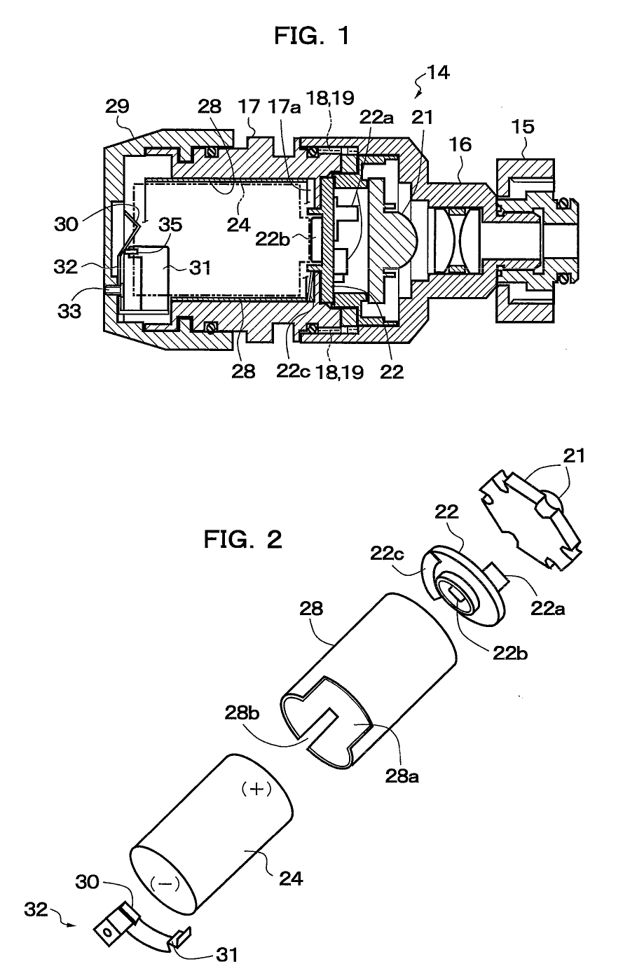

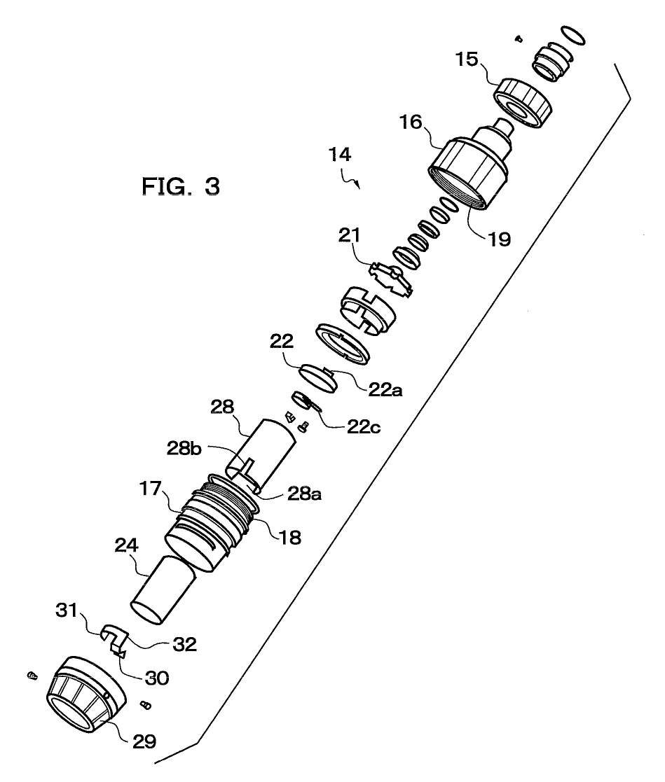

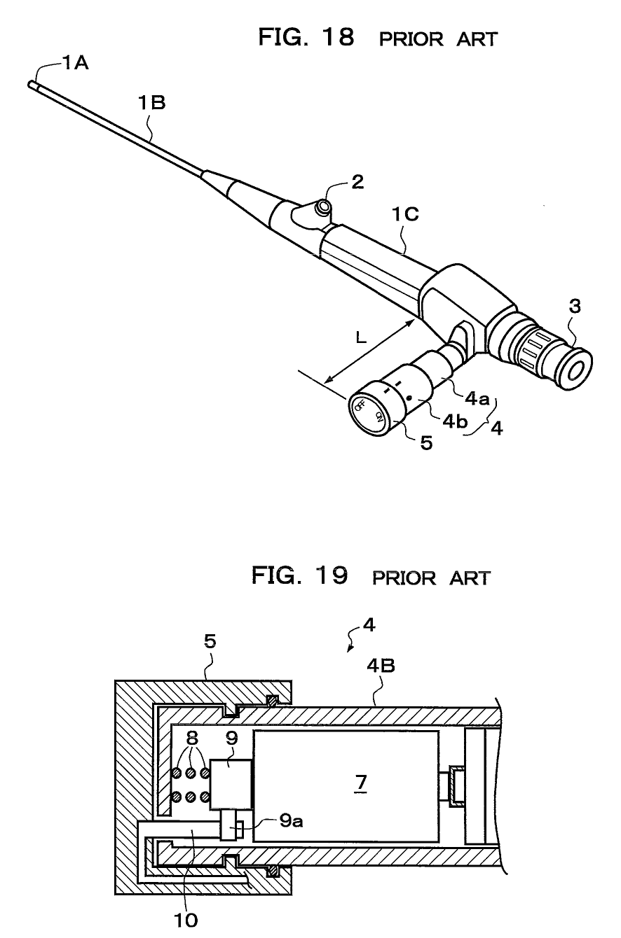

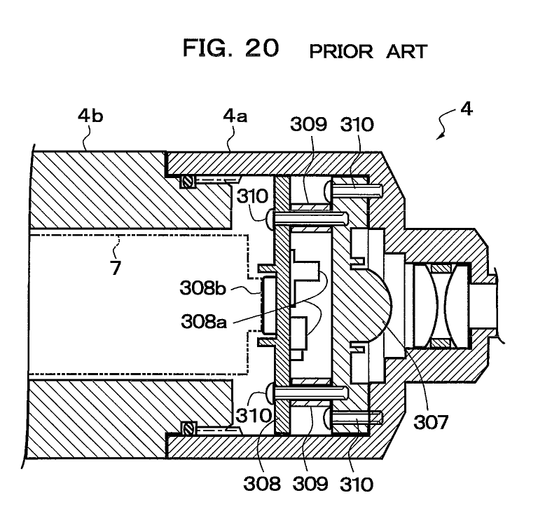

FIGS. 1 to 6 show the structure of a box for an endoscope according to a first embodiment. The first embodiment is a light source box (functional box) using a stored battery to illuminate a light source. The light source box 14 similar to the endoscope described by FIG. 18 is disposed in the endoscope operation section (1C) to project in a vertical direction from the axis of the endoscope. FIG. 1 is a sectional view of the light source box and FIG. 3 is an exploded view of each member of the light source box 14. The light source box 14 is provided with a hollow front (right side of figure) supporting body 16 having a mounting section 15 for mounting on the endoscope operation section and a hollow rear supporting body 17 having a battery storage section 17a. In other words, as shown in FIG. 3 and FIG. 5A, both components are engaged and fixed by threadable engagement of the male threaded section 18 provided on the outer peripheral tip of the rear supporting body 17 to the female threaded section 19 provided on the rear peripheral end of the front supporting body 16.

An optical lens and a circuit board 22 are disposed in the front supporting body 16. The front side face of the circuit board 22 mounts an LED section 21 mounting an LED on a fixed board and a LED drive circuit (constant current circuit or the like) 22a for driving the LED. On the rear side face of the circuit board 22, a battery connection connector 22b is provided to connect the positive (+) electrode of the battery 24 with the central section of the board and an arcuate resilient connector 22c is provided along a peripheral direction on an outer side of the connector 22b. The resilient connector 22c is connected to the negative power source line of the LED drive circuit 22a.

A battery storage section 17a is provided in the rear supporting body 17 and a cylindrical conductive body 28 is provided to function as a switch fixed conductive body on a position on an outer side of the battery 24 in the battery storage section 17a. However as shown in FIG. 2 for example, a notch 28a for placing the switch in the OFF position is provided on the rear end of the cylindrical conductive body 28 and the cylindrical outer side face of the section provided with the notch 28a functions as a sliding terminal for placing the switch in the ON position. The other notch 18b shown in FIG. 2 fixes the cylindrical conductive body 28 to the inner face of the rear supporting section 16.

A bowl-shaped rotating switch (both power input switch and light source illumination switch) 29 is rotatably provided on the rear side of the rear supporting body 17. The rotating switch (body) 29 rotates through a fixed angle required to perform ON and OFF operations by engagement of a pin towards the rotating switch 29 with an elongated groove (guide groove) formed with respect to a peripheral direction towards the rear supporting body 17. The rotating switch 29 is provided with a connector 32 which integrally forms the battery connection resilient connector 30 to which the negative (−) terminal of the battery 24 is connected and the switch movable connector (terminal) 31.

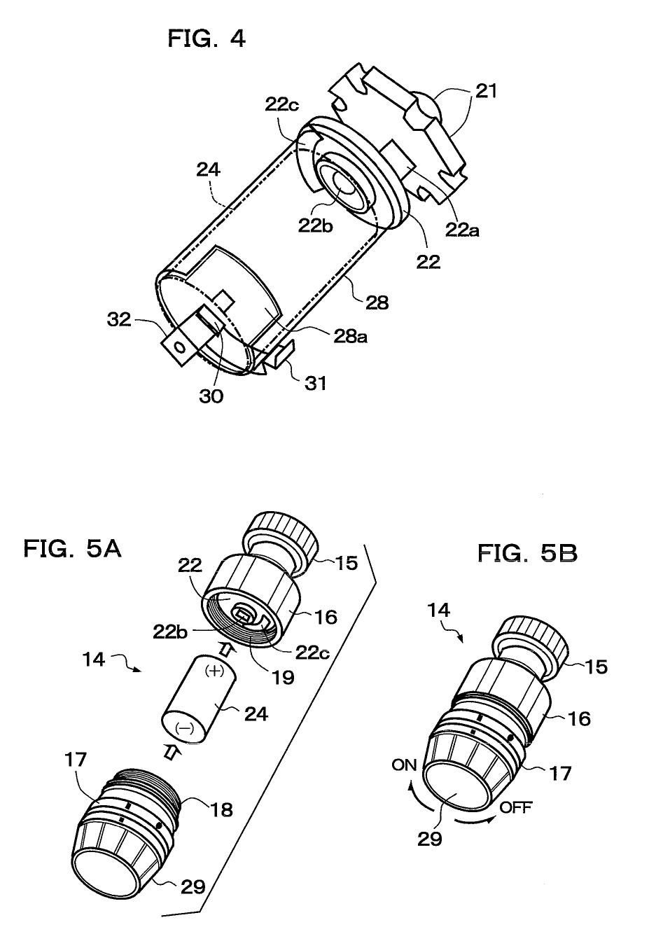

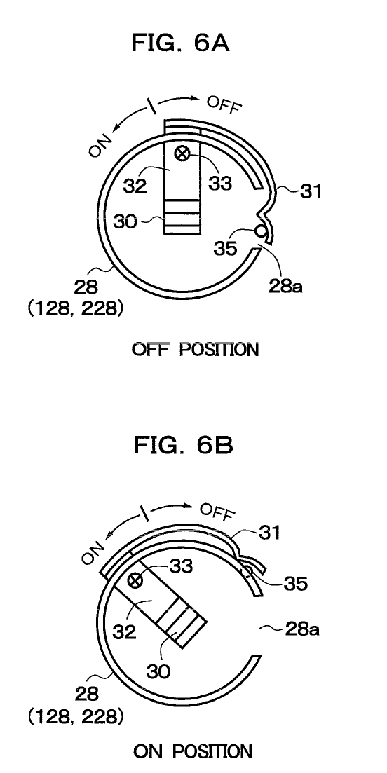

In other words, as shown in FIG. 2, FIG. 4 and FIG. 6, the resilient connector 30 is a connector in which an oblong resilient plate is curved in a dog's leg shape so that a projecting section is formed towards the negative terminal of the battery 24. The switch movable connector 31 is a connector which is formed and disposed vertically from the face of the base of the resilient connector 30 and which is bent into an arcuate shape along the inner side face (outer periphery of the cylindrical conductive body 28 or the outer periphery of the battery 24) of the battery storage section 17a. A projection projecting inwardly is formed on the distal end of the connector 31. The base of the resilient connector 30 on the connector 32 is fixed with a screw 33.

A boss (pin-shaped projection) 35 is provided on the bowl-shaped inner bottom face of the rotating switch 29 to limit the inward displacement of the switch movable connector 31. As shown in FIG. 6A, when the movable connector 31 (projecting section) displaces towards the notch 28a of the cylindrical conductive body 28, the boss 35 has the stopper function of engaging so that the movable connector 31 is not drawn inwardly, maintaining the OFF operation of the switch and avoiding contact of the movable connector 31 with the battery 24.

Since the first embodiment is arranged as described above, as shown in FIG. 1, the LED section 21 and the circuit board 22 as shown in FIG. 2 are fixed to the inner section of the front supporting body 16, the cylindrical conductive body 28 is fixed to the battery storage section 17a of the rear supporting body 17, the connector 32 is fixed to the bottom face of the rotating switch 29. Thus when the rear supporting body 17 is mounted on the front supporting body 16, the state shown in FIG. 4 results. In this state, the front side end of the cylindrical conductive body 28 makes contact with the resilient connector 22c connected to the negative power source line of the LED drive circuit 22a and the switch movable connector 31 is disposed on the outer peripheral side of the cylindrical conductive body 28.

In other words, as shown in FIG. 5A, in this light source box 14, after the battery 24 is placed into the rear supporting body 17 (battery storage section 17a), the rear supporting body 17 is mounted on the front supporting body 16 as shown in FIG. 5B by threadable engagement of the male threaded section 18 with the female threaded section 19.

When the rotating switch 29 is rotated through a fixed angle in an ON direction, the movable connector 31 fixed to the rotating switch 29 displaces from the state in the OFF position as shown in FIG. 6A to the ON state shown in FIG. 6B. In other words, FIG. 6 shows the bottom face in the rotating switch 29 as seen from the front side (opening) of the rear supporting body 17. In the OFF position as shown in FIG. 6A, the switch movable connector 31 is positioned at the notch 28a of the cylindrical conductive body 28 due to the engagement of the boss 35. When the rotating switch 29 operated to an ON position, as shown in FIG. 6B, the movable connector 31 is rotated and makes contact with the outer periphery (sliding terminal) of the cylindrical conductive body 28. In this manner, the power source switch is placed in the ON position and the LED section 21 is illuminated. In the state shown in FIG. 6B, the positional relationship is such that the movable connector 31 and the cylindrical conductive body 28 make contact prior to the movable connector 31 making contact with the boss 35.

Conversely when the rotating switch 29 is rotated to the opposite OFF position from the ON position shown in FIG. 6B, as shown in FIG. 6A, since the connector 31 displaces to the notch 28a of the cylindrical conductive body 28, the power source switch is placed in the OFF position and the LED section 21 is extinguished. When in the OFF position, the movable connector 31 is engaged by the boss 35 and in this manner, the movable connector 31 does not come into contact with the battery 24 and thus does not constitute an impediment when mounting or removing the battery 24. Although the boss 35 rotates together with the rotating switch 29, as shown in FIG. 1, it is adapted to have a length which does not reach the rear end (connector 30 side) of the cylindrical conductive body 28 and on the movable connector 31 is formed with a length which enables contact. Therefore the boss 35 has a stopper function and does not interfere with the cylindrical conductive body 28.

In the first embodiment, although the battery box was described with reference to a light source box, the light source is not limited to being driven by a battery and the invention may be applied to battery storing boxes supplying battery power to other drive sections.

### Embodiment 2

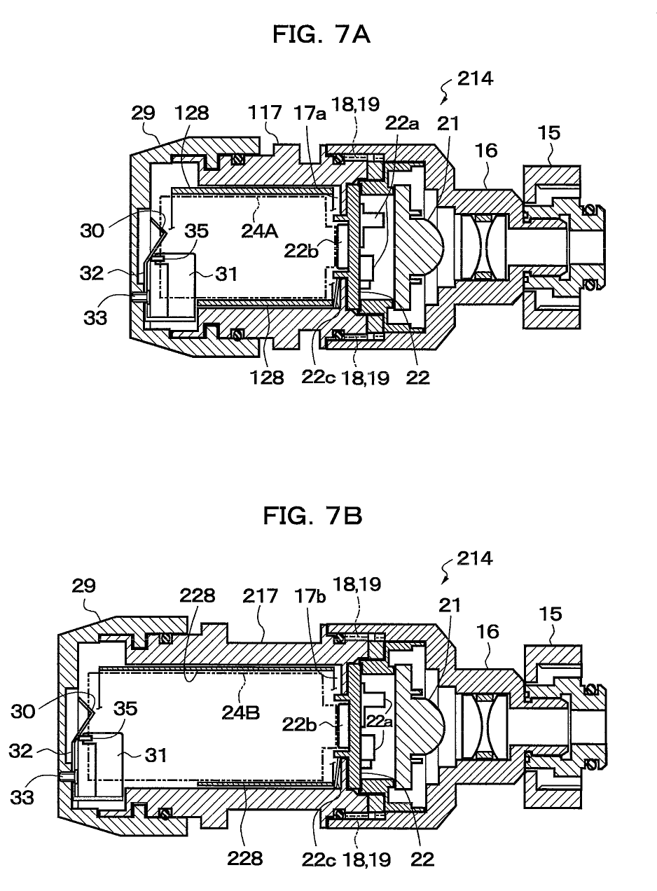

FIGS. 7A, and 7B and FIGS. 10A and 10B show two types of arrangements of an endoscope battery box according to the second embodiment. The second embodiment also relates to a light source box illuminating a light source with a stored battery. FIGS. 7A and 7B are sectional figures of two types of light source boxes 214. FIG. 2 is an exploded view of the main members peripheral to a battery in a light source box 14.

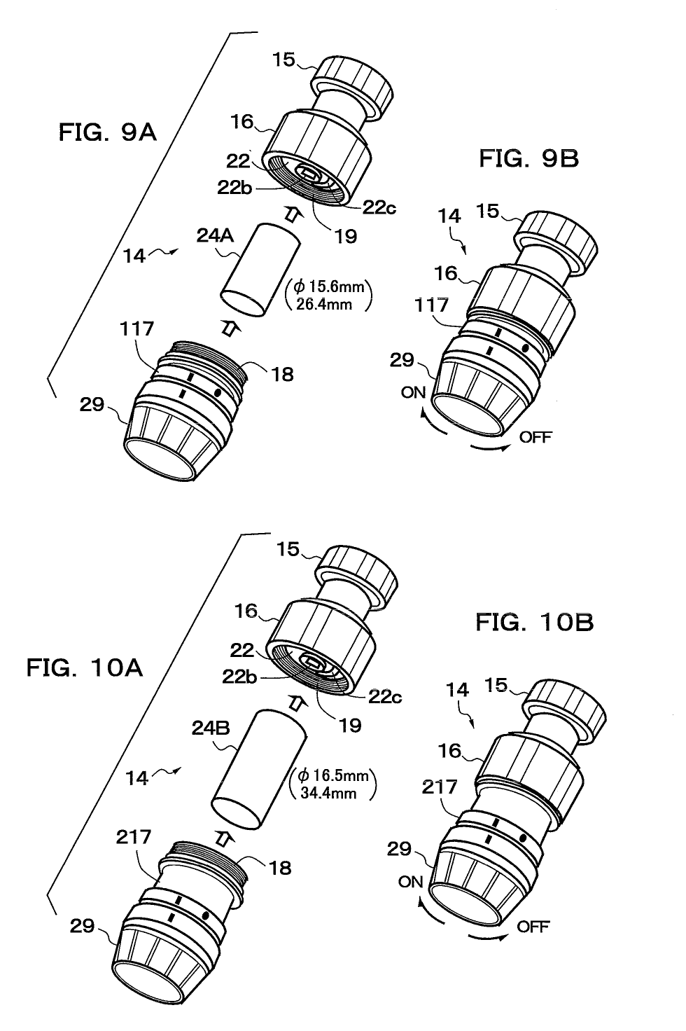

As shown in FIGS. 7A and 7B, the two types of light source box 214 in this embodiment are provided with a hollow front supporting body (common front supporting body) 16 having a mounting section 15 for mounting on the endoscope operation section and a hollow rear supporting body 117 with a battery storage section 17a and a hollow rear supporting body 217 with a battery storage section 17b which are freely detachable with respect to the front supporting body 16. In other words, as shown in FIG. 9A for example, a common (same shape) male threaded section 18 provided on an outer forward distal periphery of the rear supporting bodies 117, 217 is threadably engaged with the female threaded section 19 provided on an inner rear distal periphery of the rear supporting body 16 thereby engaging and fixing both members. Furthermore the rear supporting bodies 117 and 217 have the same wall thickness and only differ with respect to the length.

In the second embodiment, a first battery 24A which is a small type (diameter 15.6 mm, length 26.4 mm) CR2 enabling use of the endoscope for 50 minutes and a second battery 24B which is a large type (diameter 16.5 mm, length 34.4 mm) enabling use for 90 minutes are provided. The first battery 24A is stored in the battery storage section 17a of the rear supporting body 117 and the second battery 24B is stored in the battery storage section 17b of the rear supporting body 217.

A circuit board 22 is disposed on the front supporting body 16 and the front side face thereof mounts an optical lens, an LED section 21 mounting an LED and an LED drive circuit (constant current circuit or the like) 22a. On the rear side face of the circuit board 22, a battery connection connector 22b is provided to connect the positive (+) electrode of the batteries 24A, 24B with the central section of the board and an arcuate resilient connector 22c is provided with respect to a peripheral direction on an outer side of the connector 22b.

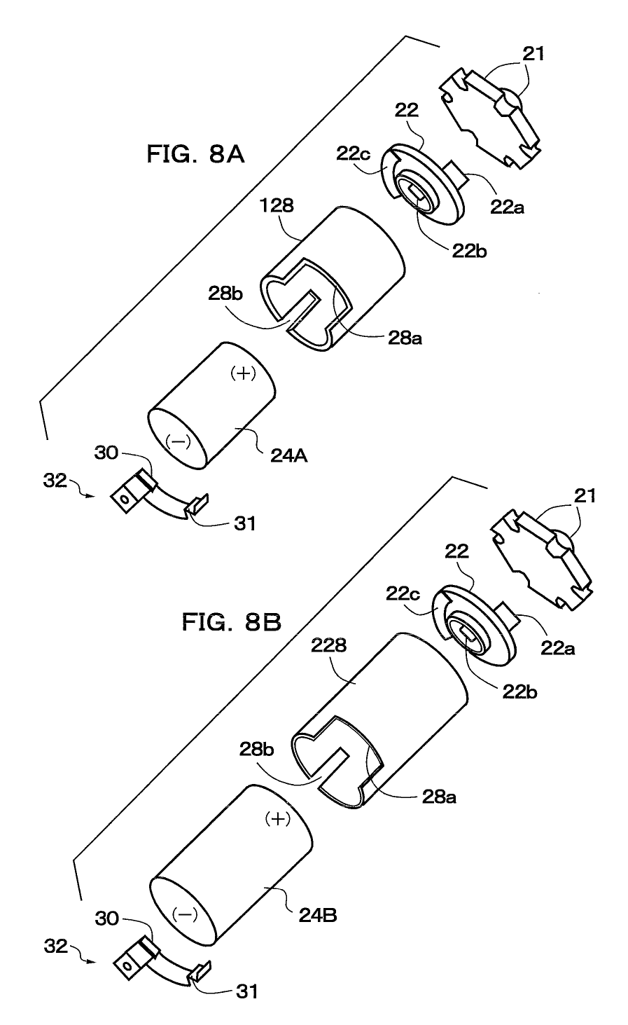

The cylindrical conductive body 128 acting a switch fixed conductive body is disposed on the rear supporting body 117 shown in FIG. 7A at a position on the outer side of the first battery 24A in the battery storage section 17a. As shown in FIG. 8A, a notch for placing the switch in the OFF position is provided on the rear end of the cylindrical conductive body 128 and the cylindrical outer side face of the section provided with the notch 28a functions as a sliding terminal for placing the switch in the ON position. The notch 28b fixes the cylindrical conductive body 128 to the inner face of the rear supporting section 16.

In the rear supporting body 217 shown in FIG. 7B, a cylindrical conductive body 228 is disposed at a position on an outer side of the second battery 24B in the battery storage section 17b. As shown in FIG. 8B, a notch 28a for switching to the OFF position and a fixing notch 28b are provided on a rear end of the cylindrical conductive body 228. The cylindrical outer face of the section provided with the notch 28a functions as a sliding terminal for placing the switch into the ON position.

In the second embodiment, the thickness of the wall of the cylindrical conductive body 128 is thicker than the cylindrical conductive body 228. In other words, as described above, since the rear supporting bodies 117 and 217 have equal wall thicknesses, adaptation to the first battery 24A and second battery 24B which have different diameters is possible by varying the wall thickness of the cylindrical conductive bodies 128, 228. That is to say, the batteries 24A, 24B which have different diameters can be firmly retained without movement in the battery storage sections 17a, 17b by increasing the wall thickness of the cylindrical conductive body 128 for the smaller first battery 24A and decreasing the wall thickness of the cylindrical conductive body 228 for the larger second battery 24B. The size of the outer periphery of the cylindrical conductive bodies 128, 228 is equal.

A bowl-shaped rotating switch (both power input switch and light source illumination switch) 29 is rotatably provided on the rear side of the rear supporting bodies 117, 217. The rotating switch 29 rotates through a fixed angle required to perform ON and OFF operations by engagement of a pin on the rotating switch 29 side with an elongated groove (guide groove) formed with respect to a peripheral direction towards the rear supporting bodies 117, 217. The rotating switch 29 is provided with a connector 32 which integrally forms the battery connection resilient connector 30 to which the negative (−) terminals of the batteries 24A, 24B is connected and the switch movable connector (terminal) 31.

In other words, as shown in FIGS. 8A and 8B, the resilient connector 30 is a connector in which an oblong resilient plate 30 is curved so that a projecting section is formed towards the negative terminal of the batteries 24A, 24B. The switch movable connector 31 is a connector which is formed and disposed vertically from the face of the base of the resilient connector 30, which is bent into an arcuate shape along the inner side face (outer periphery of the cylindrical conductive bodies 128, 228 or the outer periphery of the batteries 24A, 24B) of the battery storage sections 17a, 17b. A projection projecting inwardly is formed on the distal end of the connector 31. The base of the resilient connector 30 on the connector 32 is fixed with a screw 33.

A boss (pin-shaped projection) 35 is provided on the bowl-shaped inner bottom face of the rotating switch 29 to limit the inward displacement of the switch movable connector 31. As shown in FIG. 6A, when the movable connector 31 displaces towards the notch 28a of the cylindrical conductive body 228 (128), the boss 35 has the stopper function of engaging so that the movable connector 31 is not drawn inwardly, maintaining the OFF operation of the switch and avoiding contact of the movable connector 31 with the battery 24B (24A).

Since the second embodiment is arranged as described above, the LED section 21 and the circuit board 22 as shown in FIGS. 8A and 8B are fixed to the inner section of the front supporting body 16, the cylindrical conductive bodies 128, 228 are fixed to the battery storage sections 17a, 17b of the rear supporting bodies 117 and 217, and the connector 32 is fixed to the bottom face of the rotating switch 29. The two type of rear supporting bodies 117, 217 are mounted on the front supporting body 16.

In other words, as shown in FIG. 9A, when selecting a small capacity battery 24A, after inserting the battery 24A into the rear supporting body 117 (battery storage section 17a), as shown in FIG. 9B, the rear supporting body 117 is mounted on the front supporting body 16 by threadable engagement of the male threaded section 18 with the female threaded section 19. As shown in FIG. 10A, when selecting a large capacity battery 24B, after inserting the battery 24B into the rear supporting body 217 (battery storage section 17b), as shown in FIG. 10B, the rear supporting body 217 is mounted on the front supporting body 16 by threadable engagement of the male threaded section 18 with the female threaded section 19.

When the batteries 24A, 24B are attached in this manner, as shown in FIGS. 7A, 7B, the front end of the cylindrical conductive bodies 128, 228 makes contact with the resilient connector 22c connected to the negative power source line of the LED drive circuit 22a and furthermore the switch rotating connector 31 is disposed towards the outer peripheral side of the respective cylindrical conductive bodies 128, 228.

When the rotating switch 29 is rotated through a fixed angle in an ON direction, the movable connector 31 fixed to the rotating switch 29 displaces from the state in the OFF position as shown in FIG. 6A to the ON state shown in FIG. 6B. In this manner, the LED section 21 is illuminated. Conversely rotation of the rotating switch 29 from the ON state shown in FIG. 6B to the opposite OFF direction places the power source switch in the OFF position and the LED section 21 is extinguished.

In the second embodiment, although a method of connection by threadable engagement was described with respect to the connection of the front supporting body 16 and the rear supporting bodies 117, 217, a method of connection other than a threadable engaging structure such as resilient connection or a cam connection (bayonet) may be employed.

### Embodiment 3

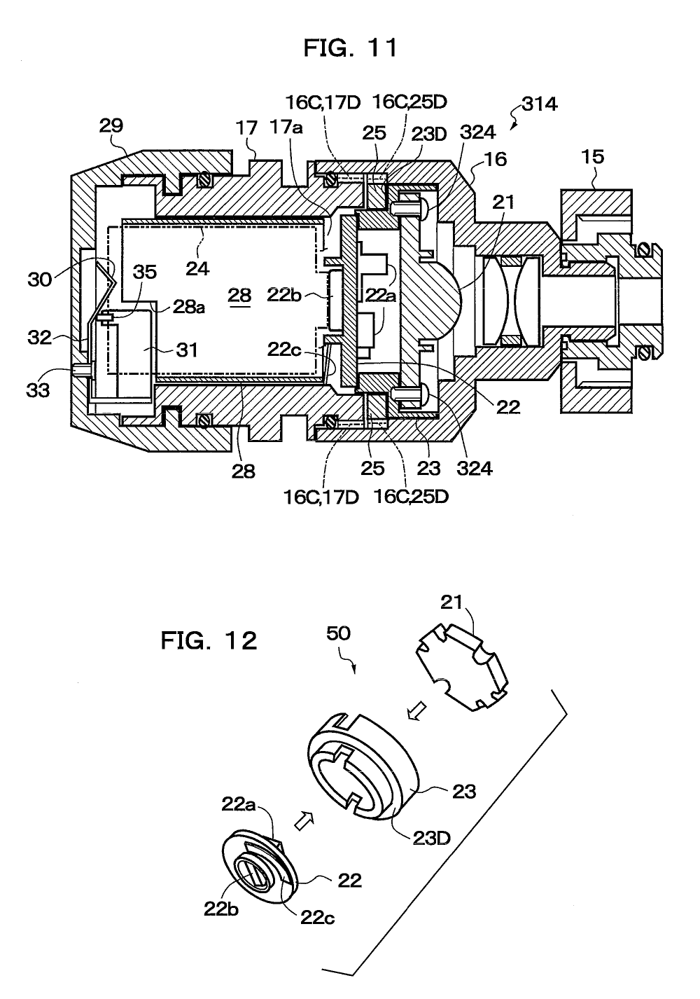

FIG. 11 to FIG. 13 show the structure of an endoscopic light source box according to a third embodiment. FIG. 11 shows a sectional view of a light source box 314. This light source box 314 in the same manner as the description above is provided with a hollow front supporting body 16 and a hollow rear supporting body 17 having a battery storage section 17a.

A circuit board 22 is disposed in the front supporting body 16. The front side face of the circuit board 22 mounts an LED section (board) 21 mounting an LED on a fixed board having a radiator plate on a rear surface and a LED drive circuit 22a including a constant current circuit or a booster circuit for driving the LED. The LED section 21 and the light source drive circuit board 22 are mounted on a holder 23 and are provided as a single assembly 50. In other words, as shown in FIG. 12, the holder 23 is constituted by a hollow cylindrical body provided with a ring-shaped step 23D in a median position by making the outer diameter of the front side large and the outer diameter of the rear side small. The LED section 21 is fixed by a fixing screw 324 into the front cylinder (base face) of the holder 23 and the front face of the light source drive circuit board 22 is fixed to the rear end face of the rear cylindrical section with an adhesive. In this arrangement, a circular groove engaging the round light source drive circuit board 22 to the rear end face of the holder 23 may be formed for the purposes of ensuring attachment and affixation.

On the rear side face of the light source drive circuit board 22, a battery connection connector 22b is provided to connect the positive (+) electrode of the battery 24 with the central section of the board and an arcuate resilient connector 22c is provided with respect to a peripheral direction on an outer side of the connector 22b. The resilient connector 22c is connected to the negative power source line of the LED drive circuit 22a.

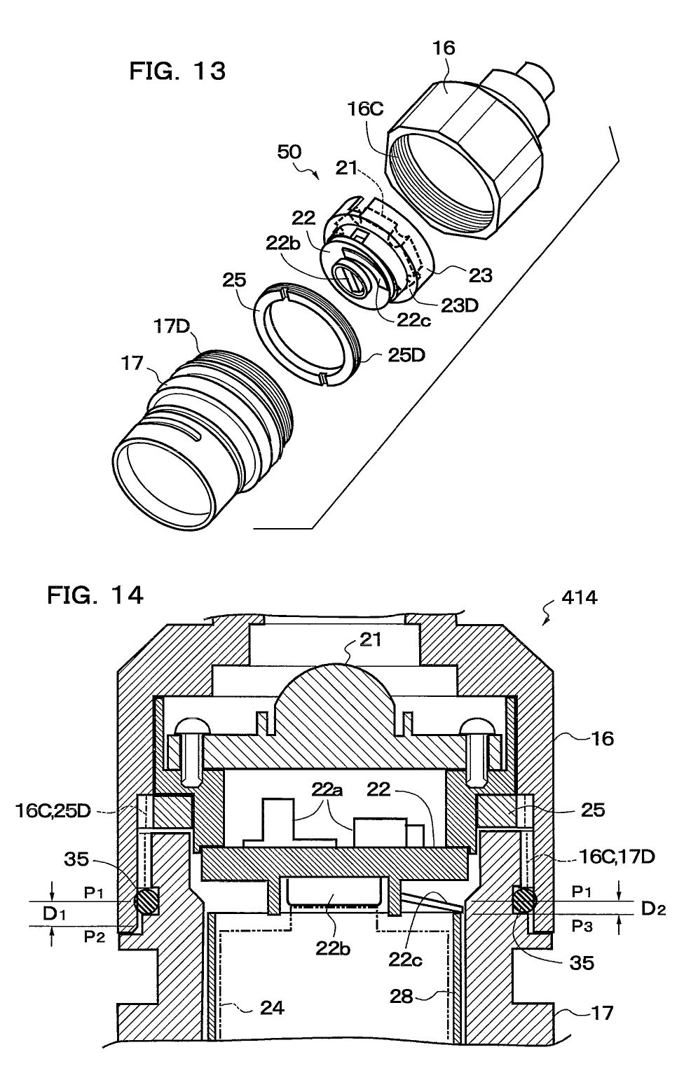

As also shown in FIG. 13, in the third embodiment, a pressing ring 25 for fixing the holder 23 is provided. The pressing ring 25 is of a size allowing abutment with the ring-shaped step (section) 23D of the holder 23 and forms a male threaded section 25D on its outer periphery. In other words, the inner diameter of the pressing ring 25 is slightly larger than outer periphery of the rear cylindrical section of the holder 23 and the outer peripheral diameter is approximately the same as the outer periphery of the front cylindrical section of the holder 23. A male threaded section 25D is formed on the outer periphery of the pressing ring 25 for threadable engagement with the female threaded section 16C formed in the inner periphery of the front supporting body 16. In the embodiment, the female section 16C of the front supporting body 16 for connecting and fixing the rear supporting body 17 is formed in a slight elongated manner to also allow use as the threadable connection section of the pressing ring 25. Consequently the structure can be simplified and the outer diameter of the light source box 314 can be reduced.

As shown above, the rear side of the holder 23 is formed with a small diameter and the pressing ring 25 is disposed on rear side of the holder 23 in order to prevent increases in the outer diameter of the front supporting body 16, in other words, the outer diameter of the light source box 314. The connection position of the rear supporting body 17 with respect to the front supporting body 16 is shifted forward in comparison to the conventional example to contribute to reducing the length (amount of projection) of the box.

In other respects, the structure of the switch cylindrical conductive body 28 in the battery storage section 17a of the rear supporting body 17 is the same as the first embodiment.

Since the third embodiment is arranged as described above, the LED section 21 as shown in FIG. 12 is fixed to the inner section of the front supporting body of the holder 23 with a screw 324 as shown in FIG. 11. The light source drive circuit board 22 is fixed with adhesive to the rear end of the rear cylindrical section of the holder 23 and the LED section 21 and the light source drive circuit board 22 are handled as a single assembly 50.

As shown in FIG. 13, after the assembly 50 is placed into the front supporting body 16, the assembly 50 is fixed and the front supporting body 16 is assembled by threadably engaging the male threaded section 25D of the pressing ring 25 with the female threaded section 16C and abutting and pressing the pressing ring 25 onto the step 23D of the holder 23. When affixation is performed by the pressing ring 25 in the above manner, the region required for connection and affixation is smaller in comparison with using a fixing screw and thus the outer diameter of the light source box 14 itself can be reduced.

The separation and engagement of the light source box 314 in the same manner as that shown in FIGS. 5A and 5B enable illumination or extinguishing of the LED section 21 in response to the operation of the rotating switch 29.

In the third embodiment, since the female threaded section 16C of the front supporting body 16 also functions as a connection section for the rear supporting body 17 of the pressing ring 25, the structure can be simplified. Furthermore the connection position of the rear supporting body 17 with respect to the front supporting body 16 is shifted forward in comparison to the conventional example by disposing the pressing ring 25 in a rear section where the holder 23 has a small diameter and thus contributes to reducing the length (amount of projection) of the light source box 14.

Although the third embodiment was described with reference to fixing the holder 23 with a pressing ring 25, the holder 23 can be fixed with a fixing screw.

### Fourth Embodiment

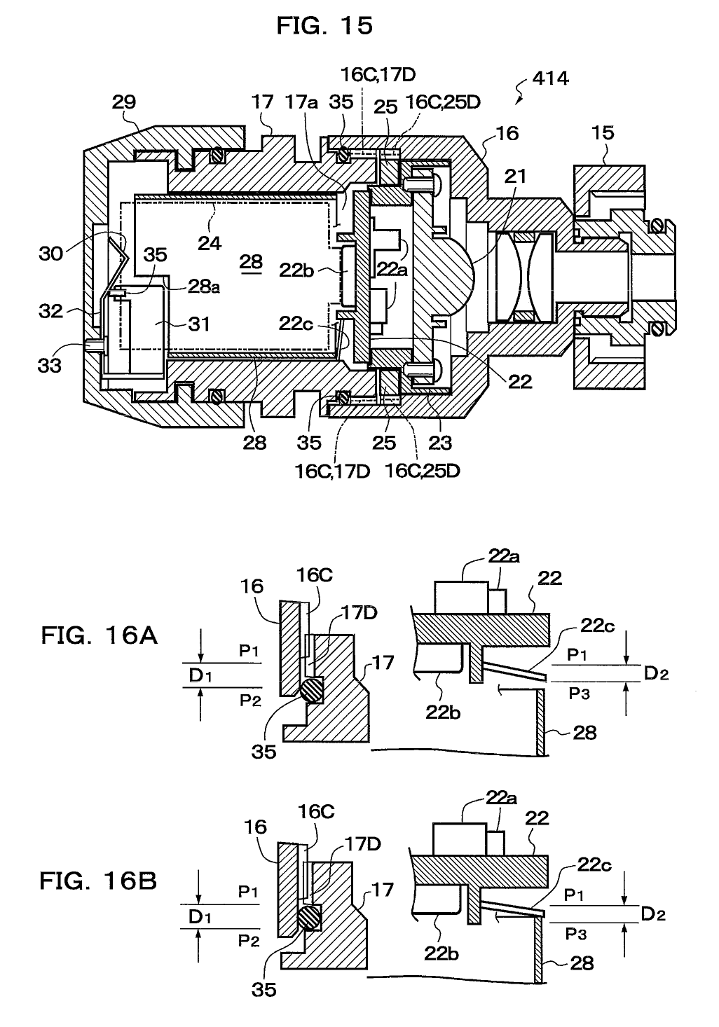

FIG. 14 to FIG. 17 show the structure of an endoscopic light source box according to a fourth embodiment. FIG. 14 shows a sectional view of a light source box 414. FIG. 15 is a sectional view of the overall light source box 414. As shown in FIG. 14, the basic structure of this light source box 314 is the same as that in the third embodiment.

In the front supporting body 16 shown in the figure, an LED section 21 mounting an LED on the front side face of a fixed board and a circuit board 22 mounting an LED drive circuit 22a on a front side face are mounted on a holder 23. A ring-shaped step 23D of the holder 23 is pressed by a pressing ring 25. Thus the light source assembly is mounted on the front supporting body 16.

On the rear side face of the light source drive circuit board 22, a battery connection connector 22b is provided to connect the positive (+) electrode of the battery 24 with the central section of the board and an arcuate resilient connector 22c is provided with respect to a peripheral direction on an outer side of the connector 22b. The resilient connector 22c is connected to the negative power source line of the LED drive circuit 22a and forms one connection point of a conductive body connection point connection structure to maintain the operation of the light source switch (hereafter rotating switch 29) in this embodiment.

As shown in FIG. 17, a cylindrical conductive body 28 is disposed and fixed connectable with the resilient connector 22c at a position on an outer side of the battery 24 in the battery storage section 17a of the rear supporting body 17. The cylindrical conductive body 28 forms another connection point for the conductive body connection point connection structure. A notch 28a for placing the switch in the OFF position is provided on the rear end of the cylindrical conductive body 28 and the cylindrical outer side face of the section provided with the notch 28a functions as a sliding terminal for placing the switch in the ON position.

Further, a rotating switch 29 is rotatably mounted on the rear side of the rear side supporting body 17, a connector 32, which integrally forms the battery connection resilient connector 30 to which the negative (−) terminal of the battery 24 is connected and the switch movable connector 31, is fixed to the rotating switch 29 with a screw 33.

A water-tight structure using an O ring 35 is provided on the engagement section of the front supporting body 16 and the rear supporting body 17 and is adapted so that commencement of the water-tight conditions by the water-tight structure is more rapid that the commencement of a connection state in connection points due to the conductive body connection structure. In other words, the O ring 35 is mounted and fixed to a groove formed on an outer periphery of the rear supporting body 17 and the O ring 35 maintains the inner section of the main body in a water-tight state by contacting with the inner peripheral face of the front supporting body 16. As shown in FIG. 14, a distance D₁ is defined as the distance to the water-tight commencement position P₂ which is forward of a final forward motion position (final position of abutment of the rear supporting body 17 with the front supporting body 16) P₁ at which the rear supporting body 17 completes forward motion due to the threadable connection of the male threaded section 17D and the female threaded section 16C, and a distance D₂ is defined as the distance to the connection point contact commencement position P₃ which is forward of the final forward motion position P₁ during connection point contact between the cylindrical conductive body 28 and the resilient connector 22c which form the conductive body connection structure. Herein the setting D₂<D₁ is performed. In this manner, after the water-tight conditions are ensured, a state of contact between connection points of the two conductive bodies is created to enable the rotating switch 29 (maintain the conductive body pathway).

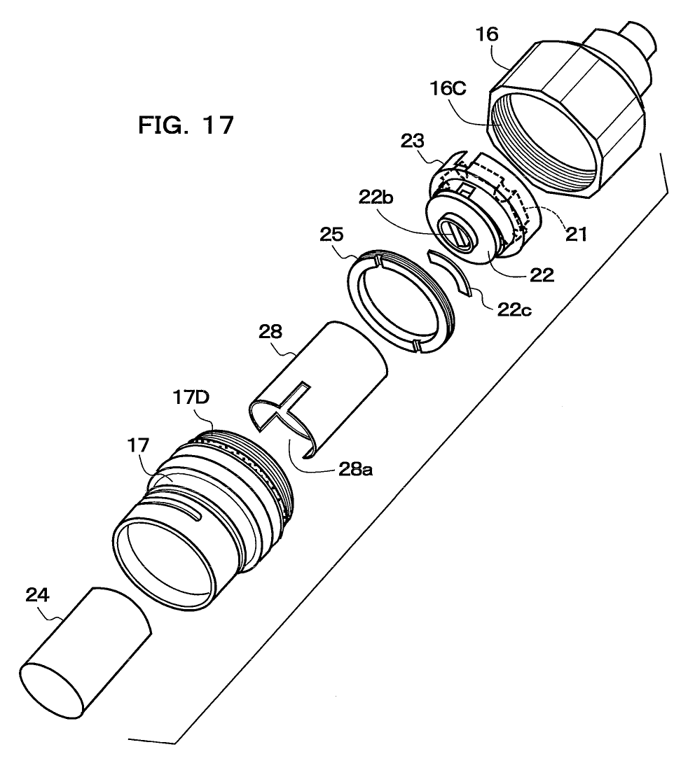

Since the fourth embodiment is arranged as described above, as shown in FIG. 16A, after the battery 24 is placed into the rear supporting body 17 of the light source box 414, the rear supporting body 17 is mounted on the front supporting body 16 as shown in FIG. 16B by threadable engagement of the male threaded section 17D with the female threaded section 16C. During mounting, since the distance D₁ from the final forward motion position P₁ to the water-tight commencement position P₂ is greater than the distance D₂ from the final forward motion position P₁ to the connection point contact commencement position P₃ ( D₂<D₁), when switch operation by the rotation switch 29 is enabled, water-tight conditions are always ensured. In other words, when water-tight conditions are not completely maintained by the connection of the front supporting body 16 and the rear supporting body 17, since the LED is not illuminated, an operator is alerted to the fact that water-tight conditions have not been maintained and can be prompted to place the connection condition in a state maintaining water-tight conditions.

FIGS. 16A and 16B show the relationship of the water-tight state above and the connection point contact state. In the fourth embodiment, as shown in FIG. 16A, when the rear supporting body 17 has moved forward relative to the front supporting body 16 and reached the water-tight commencement position P₂, the cylindrical conductive body 28 has not reached the connection point contact commencement position P₃ and, as shown in FIG. 16B, when it moves further forward past the connection point contact commencement position P₃ and comes into contact with the resilient connector 22c, the rotation switch 29 is enabled.

The separation and engagement of the light source box 414 in the same manner as that shown in FIGS. 5A and 5B performs illumination or extinguishing of the LED section 21 in response to the operation of the rotating switch 29.

In the fourth embodiment, a user engages the rear supporting body 17 and enables the rotating switch 29 to allow operation of the light source section. Therefore water-tight condition in inner sections are ensured, washing and disinfection after use can be conducted under preferred water-tight conditions and corrosion of internal electrical members is prevented.

In each embodiment above, a box was described using the example of a light source box. However the invention is not limited to driving a light source with a battery and may be applied to boxes having a function of storing batteries and supplying a battery-based power source to another drive section. Furthermore the box according to the fourth embodiment is not limited to boxes in which there is a projection in a vertical direction from the endoscope axis and can be applied to various types of boxes provided in endoscopes.

### DESCRIPTION OF SYMBOLS

1C ENDOSCOPE OPERATION SECTION
4, 14, 214, 314, 414 LIGHT SOURCE BOX
7, 24 BATTERY
16 FRONT SUPPORTING BODY
17 REAR SUPPORTING BODY
17a BATTERY STORAGE SECTION
21 LED SECTION
23 HOLDER
23D RING-SHAPED STEP
25 PRESSING RING
30 RESILIENT CONNECTOR
28 CYLINDRICAL CONDUCTIVE BODY
28a NOTCH
29 ROTATION SWITCH

### CITATION LIST

Patent Document 1: JP-A-2000-131623
Patent Document 2: JP-A-2000-56239
Patent Document 3: JP-A-2003-126034
Patent Document 4: JP-A-2003-169777

What is claimed is:

1. A box for endoscope disposed to project in a predetermined direction from an endoscope operation section and provided with a rotating switch and a battery storage section storing a battery, the box for endoscope comprising:
a resilient connector for battery connection disposed on a rear end of a box main body,
a switch movable connector connected to the resilient connector, disposed up to the inner face of the battery storage section and fixed to the rotating switch, and
a switch fixed conductive body disposed on an inner face of the battery storage section,
wherein switch operation is executed by contact/breaking contact of the switch movable connector and the switch fixed conductive body in response to the rotation operation of the rotating switch.

2. The box for endoscope according to claim 1, wherein
the switch fixed conductive body comprises a notch on a peripheral section of a conductive cylindrical body and the switch movable connector is placed in an OFF position when displaced to the notch and is placed in an ON position when contacting with the side face of the cylindrical body.

3. A box for endoscope disposed to project in a predetermined direction from an endoscope operation section and storing a battery, the box for endoscope comprising:
a box main body includes a front supporting body mounted on the operation section and a rear supporting body freely detachable from the front supporting body,
wherein the rear supporting body has a battery storage section of a size enabling the storage of respective types of batteries of differing sizes and a plurality of rear supporting bodies are provided in common with connection sections for the front supporting bodies.

4. The box for endoscope according to claim 3, wherein
a cylindrical body is provided on the rear supporting body between the battery and the side face of the battery storage section and the wall thickness of the cylindrical body is adjusted to thereby enable storage of batteries of differing diameters.

5. The box for endoscope according to claim 3, wherein
a rotating switch is provided on the rear supporting body, and
a cylindrical conductive body forming a notch on a peripheral section is fixed to the inner side of the battery storage section and a movable connector is fixed to the rotating switch, and the movable connector is placed in an OFF position when displaced to the notch and is placed in an ON position when making contact with the side face of the cylindrical conductive body, and the storage of batteries of differing diameters is enabled by adjusting the wall thickness of the cylindrical conductive body.

6. A box for endoscope disposed to project in a predetermined direction from the endoscope operation section and mounting a light source section retaining a light source for illumination and a light source drive circuit driving the light source, wherein
the light source section and the light source drive circuit are mounted on a single assembly holder and the assembly holder is fixed to the box main body.

7. The box for endoscope according to claim 6, wherein
the assembly holder is a cylindrical body forming a ring-shaped step having a smaller outer diameter on the rear side and is provided with a pressing ring forming a threaded section, and the assembly holder is engaged with the box main body by engaging with the threaded section on the supporting body side while the pressing ring abuts with the ring-shaped step.

8. A box for endoscope having a box-shaped main body, the box-shaped main body disposed in the operation section of the endoscope and comprising a base and a separating section, the box for endoscope comprising:
a water-tight structure provided on an engaging section for the separating section and the base to maintain the interior of the box-shaped main body in a water-tight state,
a connection structure for the conductive body connection points connecting two conductive body connection points to maintain an electrical path connected to the functional switch when the separating section is mounted on the base,
a connection means is provided on the engaging section to move the separating section forward and mount and fix the separating section to the base,
wherein a distance D₁ is defined as the distance to the water-tight commencement position of the water-tight structure forward of a final forward motion position at which the separating section completes forward motion with respect to the base due to the connection means, and a distance D₂ is defined as the distance to the connection point contact commencement position of the conductive body connection structure which is forward of the final forward motion position, and herein D₂<D₁.
